(19) United States
(12) Patent Application Publication
(10) Pub. No.: US 2004/0228810 A1
(43) Pub. Date: Nov. 18, 2004

(54) Sprayable sunless tanning solution with sunscreen protection

(76) Inventors: Starr Hamson, Hilton Head Island, SC (US); Henrique R. Pirotelli, Glenhaven (AU)

Correspondence Address: MERONI + MERONI, P.O. BOX 309, BARRINGTON, IL, 60011, US

(21) Appl. No.: 10/437,155

(22) Filed: May 13, 2003

(51) Int. Cl. A61K 9/00; A61K 7/42

(52) U.S. Cl. 424/47; 424/59

(57) ABSTRACT

The sprayable sunless tanning composition with sunscreen protection is an emulsion based up on phase inversion technology that enables the production of highly stable, thin water in oil emulsions. The sunscreen protection is achieved through a blend of UV A, UV B and UV C absorbers. The sunless tanning effect is achieved by the use of tanning agents such as dihydroxyacetone in conjunction with erythrulose-a keto-sugar that minimizes streaking and prolong the residence time of the tan. Spray pattern enhancers such as Alcohol are used to improve sprayability through airbrush systems that also decrease the drying time of the composition when applied to the skin. Preferably, an antioxidant ingredient such as white tea extract is also used to counter the harmful effects that ultraviolet light has on exposed skin.

### BACKGROUND OF THE INVENTION

1. Field of the Invention

The claimed invention generally relates to sunless tanning solutions. More specifically, the claimed invention relates to a sprayable sunless tanning solution with sunscreen protection.

2. Description of the Prior Art

Sunless tanning has become increasingly popular over the last several years due to increased awareness of the harmful effects of exposing skin to ultraviolet rays produced by the sun and the increased effectiveness of sunless tanning compositions available on the market. The following previously issued United States Patents disclose some of the sunless tanning compositions known in the prior art.

U.S. Pat. No. 6,447,760 issued to SaNogueira discloses a sunless tanning composition having a sunless tanning agent such as dihydroxacetone and an application indicator agent such as henna or caramel. A color fixative may be included in the composition to help prolong the effects of the sunless tanning composition.

U.S. Pat. No. 6,451,293 issued to Schreier discloses an active ingredient combination containing erythrulose and a further reducing sugar with self-tanning properties such as dihydroxyacetone for artifically tanning the skin. The compound results in an even and longer-lasting coloring of the skin than using dihydroxyacetone alone.

U.S. Pat. No. 6,482,397 issued to Scott discloses sunless-tanning compositions containing a self tanning agent, a carrier, and a coloring agent added to the composition so as to enhance the uniformity of their application to the skin.

It is important that sunless tanning compositions are evenly applied to the skin so that a natural appearing tan is achieved. Several different types of automated spray booths have been developed to evenly apply sunless tanning compositions. An example of such a spray booth is U.S. Pat. No. 6,302,122 issued to Parker that discloses an apparatus for coating a human body with a tanning composition wherein an arm with a plurality of nozzles thereon traverses within a booth to spray coat a body in the booth. Another example of a spray booth for applying sunless tanning compositions is U.S. Pat. No. 6,251,374 issued to Laughlin. The apparatus disclosed by Laughlin provides a system that atomizes the tanning composition so that it can be sprayed onto a person standing in the booth and a recovery system for collecting excess tanning composition.

These compositions and devices for applying compositions provide an alternative way to achieve the look of a suntan without exposing the skin to harmful rays produced by the sun. However, the previously conceived sunless tanning compositions have not provided a composition that can also protect the skin from harmful rays while providing the desirable effect of a sunless tan. Therefore, if a person also desired protection from harmful rays emitted by the sun, a further composition having sunscreen properties would also have to be applied to the skin.

It would be advantageous to have a sprayable sunless tanning composition that also provides sunscreen protection so that the even and thorough application of the sunless tanning composition achieved through airbrush spraying would simultaneously provide an improved way of protecting the skin from harmful rays produced by the sun. However, the prior art does not provide a sprayable sunless tanning composition with sunscreen protection. Therefore, there is a need for a composition that can provide a sunless tan while simultaneously protecting the skin from harmful rays.

### SUMMARY OF THE INVENTION

It is an object of the claimed invention to provide a sprayable sunless tanning composition with sunscreen protection equivalent to SPF 30 and higher.

It is another object of the claimed invention to provide a sprayable sunless tanning composition with sunscreen protection having improved streak resistance.

It is a further object of the claimed invention to provide a sprayable sunless tanning composition that provides sunscreen protection where the sunless tanning composition is applied.

The sprayable sunless tanning composition with sunscreen protection is an emulsion based upon phase inversion technology that enables the production of highly stable, thin water in oil emulsions. The sunscreen protection is achieved through a blend of UV A, UV B and UV C absorbers. The sunless tanning effect is achieved by the use of tanning agents such as Dihydroxyacetone (DHA) in conjunction with Erythrulose-a keto-sugar that minimizes streaking and prolong the residence time of the tan. Spray pattern enhancers such as Alcohol are used to improve sprayability through airbrush systems that also decrease the drying time of the composition when applied to the skin. Preferably, an antioxidant ingredient such as white tea extract is also used to counter the harmful effects that ultraviolet light has on exposed skin.

The claimed invention further provides a sunless tanning composition with sunscreen protection by performing a process comprising heating and agitating the wax based emulsifiers, separately heating and agitating a first portion of water, separately agitating the sunless tanning agents with the white tea extract and a second portion of water and separately blending a group of preservatives. The first portion of water is added to the emulsifiers and the mixture is then cooled. The sunless tanning agents, white tea extract, second portion of water and the preservatives are then added to the cooled mixture.

The claimed invention further provides a process for preparing a sprayable sunless tanning composition with sunscreen protection where the wax based emulsifiers are heated and agitated in a first vessel. A first portion of water is heated in a second vessel. The sunless tanning agents, antioxidant agent, and spray pattern enhancer are agitated in a third vessel. A group of preservatives are agitated in a fourth vessel. The contents of the second vessel is added to the first vessel and then cooled. The contents of the third and fourth vessel are then added to the first vessel. After a thorough mixing of the contents, the pH level and viscosity are then checked.

### DETAILED DESCRIPTION OF THE PREFERRED EMBODIMENT

The following is a brief description of the function of each of the ingredients of the preferred embodiment of the claimed invention. The claimed composition is an oil in water composition with water being used as a diluent in from about 40% to about 70% by weight of the composition.

As emulsifiers for oil-in-water and water-in-oil emulsions it is possible to use emulsifiers known per se, such as silicons, sesquioleates, sorbitan esters, alkoxylated sorbitan and fatty acid esters, alkoeylated mono-, di- and triglycerides, optionally alkoxylated polymers such as crosspolymers of ethylene oxide and propylene oxide, optionally alkoxylated fatty alcohols, fatty acids, esters of natural oil derivatives, ethers such as polyethylene glycol(n)stearyl ether and polyethylene cetyl(n)stearyl ether. Preferably, Glyceryl Stearate, ceteareth-20, cateareth-12, and cetearyl alcohol cetyl palmitate, sold as EMULGADE SE-PF from Cognis in a blended form, are used as emulsifiers in from about 5% to about 9% by weight of the composition. Ceteareth-30, sold under the name EUMULGIN B3, is also used as an emulsifer in from about 3% to about 7% by weight of the composition.

These wax-based emulsifiers were selected for their sprayability through a spray system such as an airbrush. It was found during spray testing of the solution that these preferred emulsifiers provided superior sprayability over solutions using gum based emulsifiers that tend to clog airbrush nozzles and create clumps of solution that are deposited upon the skin during application of the solution.

Solubilizers are used to solubilize the ingredients of the composition into the water. C 12-15 Alkyl benzoate, sold under the name FINSOLV TN, is used as a preferred solubilizer in from about 4% to about 9% by weight of the composition.

Previously, sunless tanning creams and lotions have been conceived providing minimal sunscreen protection, ranging in the sun protection factor range of 1 to 7. A main objective of the claimed invention is to provide a sprayable sunless tanning solution with increased sunscreen protection, providing a sun protection factor of at least 30. The preferred embodiment of the claimed invention uses the following combination and amounts of ingredients to provide superior sunscreen protection over previous sunless tanning cream and lotions. An emollient is used in the composition as a carrier for the ultraviolet absorbers. Preferably, Dicaprylyl Ether sold under the name CETIOL OE is used in from about 1% to about 4% by weight of the composition or Cetearyl Isononanoate sold under the name CETIOL SN is used in from about 1% to about 3% by weight of the composition as the emollient carrier of the absorbers.

Ultra violet absorbers are used in the composition to provide sunscreen protection to the skin when the composition is applied to the skin. There are many different ingredients that can be used as ultraviolet (UV) absorbers. Preferably, Ethylhexyl Methoxycinnamate and Methylbenzylidene Camphor are used as UV B absorbers. To achieve desirable results, the Ethylhexyl Methoxycinnamate is used in from about 7% to about 10% by weight of the composition and the Methylbenzylidene Camphor is used in from about 1% to about 2% by weight of the composition.

The composition also comprises ingredients used as UV A absorbers. Preferably, Benzophenone-3 is used in from about 2% to about 5% by weight of the composition. As an alternative to Benzophenone-3, Butyl Methoxydibenzoylmethane, available as AVOBENZONE by Givaudan-Roure, can be used in from about 1% to about 3% by weight of the composition where Benzophenone-3 is not permitted for use as an ultraviolet absorber.

In another embodiment of the invention, Octyl Methoxycinnamate, Benzophenone-3, Homomenthyl Salicylates and Octyl Salicylates are used as the ultraviolet absorbers in the compostion. Preferably, Octyl Methoxycinnamate is used in from about 7% to about 10% by weight of the composition, Benzophenone-3 is used in from about 3% to about 5% by weight of the composition, Homomenthyl Salicylates is used in about 6% to about 8% by weight of the composition and Octyl Salicylates is used in about 4% to about 5% by weight of the composition.

In a further embodiment of the invention, Octyl Methoxycinnamate, Methylbenzylidene Camphor, and Butyl Methoxydibenzoylmethane are used as the ultraviolet absorbers in the composition. Preferably, Octyl Methoxycinnamate is used in from about 7% to about 9% by weight of the composition, Methylbenzylidene Camphor is used in from about 3% to about 4% by weight of the composition, and Butyl Methoxydibenzoylmethane is used in from about 1.5% to about 3% by weight of the composition. In this embodiment, Glycerin is also used in from about 4% to about 7% by weight of the composition.

A sunless tanning agent is also used in the composition to provide the sunless tanning effect. There are many different types of agents available with Dihydroxyacetone (DHA) being a more common agent. DHA is an aliphatic ketone that reacts with proteins in the skin creating the appearance of tanned skin. Preferably, DHA is used in from about 3% to about 9% by weight of the composition as a tanning agent.

The composition also comprises a coloring agent. Erythrulose is a C4-ketosugar that penetrates lower layers of the stratum corneum when applied to the skin where it is also able to react with the free amino groups resulting in a more even and longer lasting tan. Other reducing sugars having self-tanning properties that can be used in combination with erythrulose are, for example, glucose, xylose, fructose, reose, ribose, arabinose, allose, tallose, altrose, mamose, galactose, lactose, sucrose, erythrose and glyceraldehyde. Preferably, erythrulose is used as a coloring agent in from about 1% to about 6% by weight of the composition.

The composition also comprises a stabilizer for maintaining the acidity of the composition. The DHA performs properly when the pH level is maintained at about 4.80. Preferably, citric acid is used in less than 0.5% by weight of the composition to maintain this preferred pH level. A chelating agent is also used to protect the solution from impurities in the water. Preferably, disodium EDTA is used in less than 0.1% by weight of the composition.

The composition also uses an antioxidant ingredient to protect the skin from harmful rays when the composition is applied to the skin. It has recently been discovered that white tea extract protects the skin from oxidative stress and immune cell damage that has been shown to contribute to reducing incidents of skin cancer. White tea extract is used as an antioxidant in about 0.1% to about 2% by weight of the composition with propylene glycol as a solvent to solubilize and activate the extract in the composition.

A main objective of the claimed invention is to provide a sprayable sunless tanning solution with sunscreen protection that can be dispersed through an airbrush type dispensing system. To enhance the spray pattern of the solution when being sprayed through an airbrush system, Alcohol 40 is used. It was found during testing of the solution that a solution having a high alcohol content decreased the stability of the DHA. The results of the test revealed that a range of about 2% to about 6% by weight achieved desirable results, with about 4% by weight providing the most preferred results. The alcohol also provides the desirable effect of decreasing the drying time of the solution when applied to skin.

The composition further comprises preservatives to protect the composition during nonuse. Preferably, Phenoxyethanol, Methylparaben, Ethylparaben, propylparaben and butyl paraben, sold under the name PHENONIP, are used in less than 0.3% by weight of the composition to protect the composition from bacteria. Sodium Hydroxymethylglycinate can also be used as a preservative in another embodiment of the invention in from about 0.2% to about 0.25% by weight of the composition. A solubilizer such as Octydodecanol is optionally used in less than 0.5% by weight of the compositoin to solubilize the preservatives.

IF THERE IS INFORMATION CONCERNING A PREFERRED VARIATION OF THE COMPOSITION WITH SPECIFIC PERCENTAGE AMOUNTS, THIS WOULD BE HELPFUL, BUT NOT NECESSARY. IF THERE IS INFORMATION ABOUT OTHER VARIATIONS, THIS WOULD BE HELPFUL.

The preferred embodiment of the claimed invention is preferably prepared by following the steps outlined here. However, the composition of the claimed invention can be prepared by other means.

The Glyceryl Stearate ceteareth-20 cateareth-12 cetearyl alcohol cetyl palmitate, Ceteareth-30, Cetearyl isononanoate, C 12-15 Alkyl benzoate, Ethylhexyl Methoxycinnamate, Benzophenone, Methylbenzylidene Camphor are mixed in a first suitable sized stainless steel vessel equipped with heat jacket and propeller agitation. The ingredients are then thoroughly mix toghether and heated to a temperature of from about 80 C to about 85 C.

A first portion of the water is then added into a second suitable sized stainless steel vessel equipped with heat jacket and propeller agitation. The first portion of water is then heated to 80 C to about 85 C.

The remaining water, dihydroxyacetone, erythrulose, citric acid, disodium EDTA, white tea extract, and alcohol 40 are then each sequentially added and thoroughly mixed into a third stainless steel vessel equipped with propeller agitation.

The phenoxyethanol, methylparaben, ethylparaben, propylparaben, and butyl paraben, octyldodecanol are blended thoroughly in a fourth stainless steel vessel equipped with propeller agitation.

The contents of the second vessel are added to the contents of the first vessel with brisk stirring for thorough blending. The contents of the first vessel are then cooled with stirring to just below 40 C. The contents of third vessel and fourth vessel are then added to the first vessel and briskly stirred for thorough blending, maintaining the temperature of the contents of the first vessel at about 40 C.

The pH level is then sampled and adjusted to ensure a final pH of approximately 4.80. The viscosity of the solution is then sampled with a Brookfield spindle A at approximately 25 C. to ensure that the viscosity ranges from about 900 cps to about 1400 cps. 10 rpm NEED MORE INFO HERE ABOUT HOW THE VISCOSITY RANGE WAS DETERMINED SUCH AS THE APPARATUS THAT USED THE BROOKFIELD SPINDLE.

IF THERE IS INFORMATION ABOUT DIFFERENT WAYS OF PREPARING THE COMPOSITION, THIS WOULD BE HELPFUL, BUT NOT TOTALLY NECESSARY.

Although the invention has been described by reference to some embodiments it is not intended that the novel device be limited thereby, but that modifications thereof are intended to be included as falling within the broad scope and spirit of the foregoing disclosure and the following claims.

We claim:

1. A sprayable sunless tanning composition with sunscreen protection, the composition comprising:
from about 7% to about 10% by weight ethylhexyl methoxicinnamate blended with from about 2% to about 5% by weight benzophenone-3, and from about 1% to about 4% by weight methylbenzylidene camphor providing sunscreen protection when applied to skin;
from about 0.1% to about 2% by weight white tea extract providing antioxidant protection when applied to skin;
from about 3% to about 9% by weight dihydroxyacetone providing an artificial tanning effect when applied to skin;
from about 1% to about 6% by weight erythrulose minimizing streaking of the artificial tanning effect when applied to the skin; and
from about 2% to about 6% by weight alcohol for enhancing spray pattern when sprayed through a spraying system.

2. The composition of claim 1 wherein the alcohol is Alcohol 40 at about 4% by weight of the composition.

3. A method of providing sunless tanning and sunscreen protection comprising sprayedly applying to the skin of a subject the composition of claim 1.

4. A sprayable sunless tanning composition with sunscreen protection, the composition comprising:
from about 10% to about 17% by weight of sunscreen agents, the sunscreen agents being selected from the group ethylhexyl methoxicinnamate, benzophenone-3, methylbenzylidene camphor, butyl methoxydibenzoylmethane, and the mixtures thereof;
from about 4% to about 15% by weight sunless tanning agents, the sunless tanning agents being selected from the group dihydroxyacetone, erythrulose, and the mixtures thereof; and
from about 2% to about 6% by weight alcohol for enhancing spray pattern when sprayed through a spraying system.

5. The composition of claim 4 further comprising from about 0.1% to about 2% by weight white tea extract providing antioxidant protection when applied to skin.

6. The composition of claim 4 wherein the alcohol is Alcohol 40 at about 4% by weight of the composition.

7. A method of providing sunless tanning and sunscreen protection comprising sprayedly applying to the skin of a subject the composition of claim 6.

8. A sprayable sunless tanning composition with sunscreen protection formed by the process comprising:
heating and agitating wax based emulsifiers from about 80 C to about 85 C;
separately heating and agitating a first portion of water from about 80 C to about 85 C in a second vessel;
separately adding and agitating dihydroxyacetone, erythrulose, citric acid, disodium EDTA, white tea extract and alcohol into a second portion of water;
separately blending phenoxyethanol, methylparaben, ethylparaben, propylparaben, butyl paraben, and octyldodecanol;
adding the first portion of water to the wax based emulsifiers;
cooling the first portion of water and the wax based emulsifiers below about 40 C;
adding the second portion of water, dihydroxyacetone, erythrulose, citric acid, disodium EDTA, white tea extract and alcohol 40 to the first portion of water and the wax based emulsifiers;
adding the phenoxyethanol, the methylparaben, the ethylparaben, the propylparaben, the butyl paraben and the octyldodecanol to the second portion of water, the dihydroxyacetone, the erythrulose, the citric acid, the disodium EDTA, the white tea extract, the alcohol 40, the first portion of water and the wax based emulsifiers; and
cooling the phenoxyethanol, the methylparaben, the ethylparaben, the propylparaben, the butyl paraben the octyldodecanol, the second portion of water, the dihydroxyacetone, the erythrulose, the citric acid, the disodium EDTA, the white tea extract, the alcohol 40, the first portion of water and the wax based emulsifiers below about 40 C.

9. The composition of claim 8 wherein the wax based emulsifiers comprise Glyceryl Stearate, ceteareth-20, cateareth-12, cetearyl alcohol cetyl palmitate and Ceteareth-30.

10. A process for preparing a sprayable sunless tanning composition with sunscreen protection, the process comprising:
adding wax based emulsifiers into a first vessel;
heating the wax based emulsifiers in the first vessel to from about 80 C to about 85 C;
agitating the wax based emulsifiers in the first vessel;
adding a first portion of water into a second vessel;
heating the first portion of water in the second vessel to from about 80 C to about 85 C;
agitating the first portion of water in the second vessel;
adding dihydroxyacetone, erythrulose, citric acid, disodium EDTA, white tea extract, alcohol and a second portion of water into a third vessel;
agitating the dihydroxyacetone, erythrulose, citric acid, disodium EDTA, white tea extract, alcohol and a second portion of water in the third vessel;
adding phenoxyethanol, methylparaben, ethylparaben, propylparaben, butyl paraben, and octyldodecanol into a fourth vessel;
adding contents of the second vessel to contents of the first vessel;
agitating the contents of the first vessel;
cooling the contents of the first vessel below about 40 C;
adding contents of the third vessel and fourth vessel to the contents of the first vessel;
adjusting temperature of the contents of the first vessel to about 40 C;
sampling pH level of the contents in the first vessel;
adjusting the pH level of the contents in the first vessel to about 4.80.

11. The process of claim 10 wherein the step of adding wax based emulsifiers into the first vessel comprises adding Glyceryl Stearate, ceteareth-20, cateareth-12, and cetearyl alcohol cetyl palmitate.

12. A sprayable sunscreen composition with sunless tanning properties, the composition comprising:
from about 10.5% to about 16% by weight of sunscreen agents, the sunscreen agents being selected from the group octyl methoxycinnamate, methylbenzylidene camphor, butyl methoxydibenzoylmethane, and the mixtures thereof;
from about 4% to about 15% by weight sunless tanning agents, the sunless tanning agents being selected from the group dihydroxyacetone, erythrulose, and the mixtures thereof; and
from about 0.1% to about 2% by weight white tea extract providing antioxidant protection when applied to skin.

13. The composition of claim 12 further comprising from about 2% to about 6% by weight alcohol for enhancing spray pattern when sprayed through a spraying system.

14. The composition of claim 13 wherein the alcohol is at about 4% by weight of the composition.

15. A method of providing sunless tanning and sunscreen protection comprising sprayedly applying to the skin of a subject the composition of claim 12.

16. A sprayable sunscreen composition with sunless tanning properties, the composition comprising:
from about 20% to about 28% by weight sunscreen agents, the sunscreen agents being selected from the group octyl methoxycinnamate, benzophenone-3, homomenthyl salicylates, octyl salicylates, and the mixtures thereof;
from about 4% to about 15% by weight sunless tanning agents, the sunless tanning agents being selected from the group dihydroxyacetone, erythrulose, and the mixtures thereof;
from about 0.1% to about 2% by weight white tea extract providing antioxidant protection when applied to skin; and
from about 2% to about 6% by weight alcohol for enhancing spray pattern when sprayed through a spraying system.
